(19) United States
(12) Patent Application Publication
(10) Pub. No.: US 2002/0058640 A1
(43) Pub. Date: May 16, 2002

(54) Liquid based vaso-occlusive compositions

(75) Inventors: Robert M. Abrams, Sunnyvale, CA (US); Paul C. Slaikeu, Hayward, CA (US); James J. Barry, Marlborough, MA (US); Joseph C. Eder, Los Altos Hills, CA (US); Michael P. Wallace, Pleasanton, CA (US)

Correspondence Address: LYON & LYON LLP, 633 WEST FIFTH STREET, SUITE 4700, LOS ANGELES, CA, 90071, US

(73) Assignee: SCIMED Life Systems, Inc., Maple Grove, MN (US)

(21) Appl. No.: 10/038,730

(22) Filed: Jan. 2, 2002

Related U.S. Application Data
(63) Continuation-in-part of application No. 09/351,769, filed on Jul. 12, 1999.

(51) Int. Cl. A61K 48/00; A61K 38/06

(52) U.S. Cl. 514/44; 514/18

(57) ABSTRACT

This relates to a composition for forming a biologically active anatomical occlusion typically within the vasculature of a patient. More particularly, it concerns an occlusive agent which may be made from a precursor composition containing at least one biodegradable, polymeric component and at least one biologically active agent. The occlusive agent may further include solid or dissolved radio-opacifiers and known vaso-occlusive devices. The resulting occlusion, a bioactive solid, encourages cellular attachment and growth while maintaining favorable handling, deployment, and visualization characteristics.

### FIELD OF THE INVENTION

This invention relates to compositions for forming a biologically active anatomical occlusion typically within the vasculature of a patient. More particularly, it concerns an occlusive agent which may be made from a precursor composition containing at least one biodegradable, polymeric component and at least one biologically active agent. The occlusive agent may further include solid or dissolved radio-opacifiers and known vaso-occlusive devices. The resulting occlusion, a bioactive solid, encourages cellular attachment and growth while maintaining favorable handling, deployment, and visualization characteristics.

### BACKGROUND

This invention relates to liquid-based polymeric compositions, or occludant precursors, that may be injected via a catheter to form occlusions in a selected body region. In particular, the resulting bioactive solid materials may be used to block blood flow in portions of malfunctioning human organs such as the kidney, spleen, and liver, or to block blood flow into the malfunctioning areas of blood vessels such as arterio-venous malformations (AVM) and aneurysms.

The artificial blocking of blood flow is known generically as “embolization.” The embolization of a vessel in an organ may be used to treat a variety of maladies; typically, though, embolization is used: 1) to control the bleeding caused by trauma, 2) to prevent profuse blood loss during an operation requiring dissection of blood vessels, 3) to obliterate a portion of or a whole organ having a tumor, or 4) to block the blood flow into abnormal blood vessel structures such as AVM's and aneurysms.

There are a variety of materials and devices which have been used for embolization. These include platinum and stainless steel microcoils, polyvinyl alcohol sponges (Ivalone), and cyanoacrylate glues (n-butyl and iso-butyl cyanoacrylate glue). See, Interventional Radiology, Dandlinger et al, ed., Thieme, N.Y., 1990:295-313. Of these, the cyanoacrylate glues have an advantage over other embolic materials in ease of delivery in that they are the only liquid embolics currently available to neurosurgeons. However, the constituent cyanoacrylate polymers have the disadvantage of being biodegradable. The degradation product, formaldehyde, is highly toxic to the neighboring tissues. See, Vinters et al, “The Histotoxocity of Cyanoacrylate: A Selective Review”, Neuroradiology 1985; 27:279-291. Another disadvantage of cyanoacrylate materials is that the polymer will adhere both to the blood vessel and to the tip of the catheter. Thus physicians must retract the catheter immediately after injection of the cyanoacrylate embolic material or risk adhesion of the cyanoacrylate and the catheter to the vessel.

Another class of liquid embolic materials—precipitative materials—was invented in late 80's. See, Sugawara et al, “Experimental Investigations Concerning a New Liquid Embolization Method: Combined Administration of Ethanol-Estrogen and Polyvinyl Acetate”, Neuro Med Chir (Tokyo) 1993; 33:71-76; Taki et al, “A New Liquid Material for Embolization of Arterio-Venous Malformations”, AJNR 1990:11:163-168; Mandai et al, “Direct Thrombosis of Aneurysms with Cellulose Acetate Polymer. Part I: Results of Thrombosis in Experimental Aneurysms.” J. Neurosurgery 1992; 77:493-500. These materials employ a different mechanism in forming synthetic emboli than do the cyanoacrylate glues. Cyanoacrylate glues are monomeric and rapidly polymerize upon contact with blood. Precipitative materials, on the other hand, are pre-polymerized chains that precipitate into an aggregate upon contact with blood.

In the precipitation method, the polymer is dissolved in a solvent that is miscible with blood, and upon contact with that blood, the solvent is diluted and the water-insoluble polymer precipitates. Ideally, the precipitate forms a solid mass and thus occludes the vessel.

One such precipitative material used in this way was polyvinyl acetate (PVAc). Takahashi et al. dissolved the polymer in an ethanol/water mixture and delivered the mixture to an AVM for embolization. Also, poly(ethylene-co-vinyl alcohol) (“EVAL”) and cellulose acetate (CA) dissolved in 100% DMSO have also been used in clinical procedures. See, Taki et al, “A New Liquid Material for Embolization of Arterovenous Malformations”, AJNR 1990; 11: 163-168 and Mandai et al, “Direct Thrombosis of Aneurysms with Cellulose Polymer: Part I: Results of Thrombosis in Experimental Aneurysms”, J. Neurosurgery 1992; 77:493-500.

Polymeric materials such as polysiloxanes, ethylene vinyl alcohol, cellulose acetates, hydrogels, polyacrylonoitriles, nitrocellulose, polyvinyl acetates, urethane and styrene/maleic acid have been used, typically in conjuctions with solvents. (see, e.g., U.S. Pat. No. 4,795,741 to Leshchiner et al; U.S. Pat. No. 4,551,132 to Pasztor et al; U.S. Pat. No. 5,403,278 to Ernst et al; U.S. Pat. No. 5,580,568 to Greffet al; U.S. Pat. No. 5,667,767 to Greff et al; U.S. Pat. No. 5,695,480 to Evans et al; U.S. Pat. No. 5,702,361 to Evans et al; U.S. Pat. No. 5,830,178 to Jones et al; and U.S. Pat. No. 5,851,508 to Greff et al).

One potential problem in using the precipitating polymers mentioned above is the use of organic solvents to dissolve the polymers, i.e., ethanol for PVAc and DMSO for EVAL and CA. These materials are strong organic solvents that can dissolve the catheter hub, and, in the case of DMSO, can damage microcapillary vessels and surrounding tissues. These solvents are also known to cause vasospasm of blood vessels. Although PVAc is soluble in solvents which are milder than those needed for dissolution of EVAL or CA, a PVAc solution has a problem of its own: its radio-opacity is very low, i.e., the contrast concentration is only 100 mg I/ml equivalent.

Injectable materials such as microfibrillar collagen, various polymeric foams and beads have also been used. (see, e.g., U.S. Pat. No. 5,823,198 to Jones et al).

Other available vaso-occlusive devices include mechanical vaso-occlusive devices. Examples of such devices are helically wound coils, ribbons and braids. Various shaped coils have been described. For example, U.S. Pat. No. 5,624,461 to Mariant describes a three-dimensional in-filling vaso-occlusive coil. U.S. Pat. No. 5,639,277 to Mariant et al. describe embolic coils having twisted helical shapes and U.S. Pat. No. 5,649,949 to Wallace et al. describes variable cross-section conical vaso-occlusive coils. A random shape is described, as well. U.S. Pat. No. 5,648,082 to Sung et al., describes methods for treating arrhythmia using coils which assume random configurations upon deployment from a catheter. U.S. Pat. No. 5,537,338 describes a multi-element intravascular occlusion device in which shaped coils may be employed. U.S. Pat. No. 5,826,587 entitled “Ultrasoft Embolization Coils with Fluid-Like Properties” by Berenstein et al., describes a coil having little or no shape after introduction into the vascular space.

There are a variety of ways of discharging shaped coils and linear coils into the human vasculature. In addition to those patents which apparently describe only the physical pushing of a coil out into the vasculature (e.g., Ritchart et al.), there are a number of other ways to release the coil at a specifically chosen time and site. U.S. Pat. No. 5,354,295 and its parent, U.S. Pat. No. 5,122,136, both to Guglielmi et al., describe an electrolytically detachable embolic device. Mechanically detachable devices are also known, as in for instance, U.S. Pat. No. 5,234,437, to Sepetka; U.S. Pat. No. 5,250,071, to Palermo; U.S. Pat. No. 5,261,916, to Engelson, and U.S. Pat. No. 5,304,195, to Twyford et al.

In other attempts to increase thrombogenesis, vaso-occlusive coils have also been treated with variety of substances. For instance, U.S. Pat. No. 4,994,069, to Ritchart et al., describes a vaso-occlusive coil that assumes a linear helical configuration when stretched and a folded, convoluted configuration when relaxed. The stretched condition is used in placing the coil at the desired site (by its passage through the catheter) and the coil assumes a relaxed configuration—which is better suited to occlude the vessel—once the device is so placed. Ritchart et al. describes a variety of shapes. The secondary shapes of the disclosed coils include “flower” shapes and double vortices. The coils may be coated with agarose, collagen, or sugar.

U.S. Pat. No. 5,669,931 to Kupiecki discloses coils that may be filed or coated with thrombotic or medicinal material. U.S. Pat. No. 5,749,894 to Engleson discloses an aneurysm closure method which involves a reformable polymer.

U.S. Pat. No. 5,536,274 to Neuss shows a spiral implant which may assume a variety of secondary shapes. Some complex shapes can be formed by interconnecting two or more of the spiral-shaped implants. To promote blood coagulation, the implants may be coated with metal particles, silicone, PTFE, rubber latexes, or polymers.

None of these documents disclose a vaso-occlusive precursor comprising a biodegradable polymer and at least one bioactive material nor the resulting biodegradable, bioactive polymeric vaso-occlusion produced in situ from the inventive precursor composition.

### SUMMARY OF THE INVENTION

As noted above, this invention involves a polymeric mixture or occlusive precursor comprising a dissolved or reactable biodegradable polymer and at least one bioactive material in a biologically tolerated solvent-containing solution. The polymeric mixture or occlusive precursor either precipitates on contact with water or water-containing liquids such as blood or reacts in the body to form an inventive, bioactive occlusive mass. Preferably, the solvent is a solvent such as ethanol because dilute ethanol has only minor toxic or harmful effects to the human body when compared to other organic solvents.

The present invention further preferably includes a polymeric precursor or resulting composition containing an x-ray contrast agent. Preferably, the composition preferably contains as much x-ray contrast agent as possible so that the injection of the inventive composition to the selected site in the body through a long catheter is visible under x-ray fluorometry and thus the injection is controllable.

In addition, the invention includes a procedure for introducing both the inventive and related solutions into the human body to form the resulting inventive embolic occlusion masses. Finally, the invention includes a procedure for introduction of the inventive polymeric mixture or occlusive precursor into or with a mechanical occlusive device such as a coil or braid.

### DESCRIPTION OF THE INVENTION

This invention includes a composition of matter which may be considered an occlusive agent precursor and the resulting occlusive material. The invention may be used to occlude selected sites within the body. Specifically, the precursor composition comprises a mixture or solution of: a.) at least one polymer-forming or dissolved polymeric biodegradable material, b.) at least one biologically active material, preferably a medicine or angiogenic material, and c.) a pharmaceutically acceptable carrier solvent. The carrier solvent is selected so that it dissolves the polymer-forming or dissolved polymeric biodegradable material and the one biologically active materials, is acceptable for introduction into the human body with a minimum of side effects, and upon contact with blood or other body fluids either allows the dissolved polymeric biodegradable material to precipitate from solution to form inventive occlusive aggregates of the polymer or permits the polymer-forming material to form a mass. The inventive compositions may also contain a dissolved or suspended radio-opacifier.

Generalized methods for introducing this inventive composition and related compositions into the human body with or without other mechanical occlusive devices also form an aspect of this invention.

Polymers

Preferred polymers are biodegradable and those that are sufficiently hydrophobic to balance an amount of hydrophilicity on the polymer chain such that the polymer is dissolved in the precursor composition but precipitates from the composition when the precursor composition is diluted by, e.g., blood or saline solutions. Hydrophilicity can be increased via the presence of, e.g., alcoholic groups in the chain. If the hydrophilicity of the polymer is increased too far, however, and too many alcoholic groups are introduced, the polymer itself becomes soluble in blood and thus does not effectively function as an embolic material. Conversely, if the hydrophobicity of the polymer is not controlled, the polymer is not sufficiently soluble in solvents which are both miscible in blood and safe for use in the human body.

Optimum polymers which have both the appropriate solubility and the biodegradability include biodegradable polyesters such as polyglycolic acid, polylactic acid, polycaprolactone, and their copolymers as well as polyhydroxybutyrate and polyhydroxyvalerate and their copolymers as well as copolymers with trimethylene and the family of polyemhydrides. Other polymers which are generally suitable are those polymers used to form dissolvable sutures for the human body.

Bioactive Materials

Non-limiting examples of bioactive materials which increase cell attachment and/or thrombogenicity include both natural and synthetic compounds, e.g., collagen, fibrinogen, vitronectin, other plasma proteins, growth factors (e.g., vascular endothelial growth factor, “VEGF”), synthetic peptides of these and other proteins having attached RGD (arginine-glycine-aspartic acid) residues, generally at one or both termini, or other cell adhesion peptides, i.e., GRGDY, oligonucleotides, full or partial DNA constructs, natural or synthetic phospholipids, or polymers with phosphorylcholine functionality. In addition, polynucleotide sequences encoding peptides (e.g., genes) involved in wound healing or promoting cellular attachment may also be used. Other components having a specific role may be included, e.g., genes, growth factors, biomolecules, peptides, oligonucleotides, members of the integrin family, RGD-containing sequences, oligopeptides, e.g., fibronectin, laminin, bitronectin, hyaluronic acid, silk-elastin, elastin, fibrinogen, and other basement membrane proteins with bioactive agents.

Other bioactive materials which may be used in the present invention include, for example, pharmaceutically active compounds, proteins, oligonucleotides, ribozymes, anti-sense genes, DNA compacting agents, gene/vector systems (i.e., anything that allows for the uptake and expression of nucleic acids), nucleic acids (including, for example, naked DNA, cDNA, RNA, DNA, cDNA or RNA in a noninfectious vector or in a viral vector which may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic polymers that are selected from a number of types depending on the desired application, including retrovirus, adenovirus, adeno-associated virus, herpes simplex virus, and the like. For example, biologically active solutes include antithrombogenic agents such as heparin, heparin derivatives, urokinase, PPACK (dextrophenylalanine proline arginine chloromethylketone), rapamycin, probucol, and verapimil; angiogenic and anti-angiogenic agents; anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-mitotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directly against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms, and combinations thereof.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides of the invention can also code for therapeutic polypeptides. A polypeptide is understood to be any translation production of a polynucleotide regardless of size, and whether glycosylated or not. Therapeutic polypeptides include as a primary example, those polypeptides that can compensate for defective or deficient species in an animal, or those that act through toxic effects to limit or remove harmful cells from the body. In addition, the polypeptides or proteins that can be incorporated into the polymer composition, or whose DNA can be incorporated, include without limitation, proteins competent to induce angiogenesis, including factors such as, without limitation, acidic and basic fibroblast growth factors, vascular endothelial growth factor (including VEGF-2, VEGF-3, VEGF-A, VEGF-B, VEGF-C) hif-1 and other molecules competent to induce an upstream or downstream effect of an angiogenic factor; epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; thymidine kinase (“TK”) and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

In one example of the present invention, the inventive composition has recombinant nucleic acid incorporated therein, wherein the recombinant nucleic acid comprises a viral vector having linked thereto an exogenous nucleic acid sequence. “Exogenous nucleic acid sequence” is used herein to mean a sequence of nucleic acids that is exogenous to the virus from which the vector is derived. The concentration of the viral vector, preferably an adenoviral vector, is at least about 10¹⁰ plaque forming units (“p.f.u.”), preferably at least about 10¹¹ p.f.u. Alternatively, the concentration of the viral vector is limited by the concentration that results in an undesirable immune response from a patient.

The bioactive agents may further contain additional materials which have one or more functions, including, but not limited to, providing a therapeutic for local or blood borne delivery, or enhancing thrombosis, coagulation, or platelet activity.

Solvent Systems

An appropriate polymer is dissolved in a suitable solvent for use as an occludant precursor. Appropriate solvents are biologically tolerated or pharmaceutically acceptable in nature and are typically polar, substantially non-toxic, and water miscible. Various suitable alcohols, ethers, amides, and glycols and their mixtures with each other or with water will be apparent to the worker of ordinary skill in this art. In general, the solvent or solvent system must be able to completely dissolve the chosen polymer and the biologically active agent and then upon introduction of that solution to a mammalian site containing an aqueous medium (naturally occurring or artificially introduced) allow the dissolved polymer to fall out of solution and form an agglomerate. Although many of these generically provided solvent systems would be suitable in certain situations where strong solvents would accelerate the occlusive activity of the polymer, e.g., where denaturing localized tissue would enhance the ultimate activity of causing tumor atrophy, an especially desirable solvent system is a mixture of ethanol and water.

Embolic Agent Precursor

Because these inventive compositions are desirably used in regions of the vasculature which are both very tortuous and in which the vessel lumen are very narrow, the catheters through which these compositions are placed must be quite small. To allow ease of injection and to minimize the danger of immobilizing normal vessels around the desired treatment site, the viscosity of the inventive solution should be minimized, consistent with the other requirements noted herein.

Because the viscosity of a polymer solution is very sensitive to polymer molecular weight (MW_{w}), particularly at high polymer concentration, the MW_{w} of the polymer should typically be less than about 500,000. However, when the MW decreases, the polymer becomes increasingly soluble in water. Therefore, it is desirable for the polymer to have a MW at least about than 10,000. The desired range is 10,000 to 500,000. The preferable MW is in the range of 50,000-100,000.

The concentration of polymer also typically affects both the viscosity of the solution as well as the precipitation behavior of the polymer. Principally because high polymer concentration, polymer solutions exhibit high viscosity and hence are quite unwieldy, lower concentrations, e.g., less than 30% depending upon the chosen polymer, are preferred for immobilization. If the polymer concentration is lower, the polymer occlusive mass may fragment into small pieces when introduced into the bloodstream due to high stress from the blood flow. There is an increased chance for the precipitated polymer to pass the malformation site and to end up in the lungs. About 5-50% polymer solutions are suitable for embolization. That is to say that “weight % polymer” is calculated based on the overall solution content (solvent, water, diluents, radio-opacifiers, etc.).

In some instances, a small amount of a commercial buffer (pH 7) may be desirable.

Aqueous ethanolic solutions having higher concentrations of ethanol and the chosen polymers are able to dissolve higher loads of radio-opacifiers such as metrizamide (see, U.S. Pat. No. 3,701,771) or iopromide (see, U.S. Pat. No. 4,364,921). Metrizamide is sold in a dilute form as “Amipaque” by Winthrop-Breon Laboratories, a division of Sterling Drug Inc. lopromide is often sold in a dilute form under the tradename “Ultravist”.

And, of course, radio-opacity may be enhanced by incorporating insoluble agents such as metal powders and salts of radio-opaque metals.

Methods of Use

Although the methods of using this inventive solution have been mentioned in passing above, additional description of preferred procedures may be found below. Generally speaking, the inventive precursor is introduced into the body in the following way. A catheter is introduced via usual procedures to a chosen site in a mammalian body. The site may be, e.g., a Fallopian tube, a ureteral or bile duct, a vascular site, etc. There are known devices for accessing each such site. Because of the viscosity of the solution, it is generally desirable to utilize the largest ID catheter practical in approaching the chosen site. The bolus of precursor material is then introduced into the catheter and injected into the chosen site. Because the polymer becomes nonsoluble and forms the occluding mass via the step of diluting its surroundings with an aqueous material, e.g., blood, the precursor should be introduced slowly so to form an aggregate near the catheter distal tip. More than one injection of precursor is possible using this technique. Once the mass is formed, the catheter is removed.

As noted above, it is often desirable to introduce the inventive precursor into the chosen body site along with a mechanical occlusive device such as a coil or braid. Several of these mechanical occlusive devices are described above in “The Background of the Invention.” Preferably, because of their history of safe usage and their ready availability, the device is a helically wound coil often wound into a secondary shape of some type. Such devices are often made of a radio-opaque, biocompatible material such as a metal or a polymer. Suitable metals may be selected from gold, rhenium, platinum, palladium, rhodium, ruthenium, various stainless steels, tungsten, and alloys thereof. The preferred alloy is one comprising upwards of 90% platinum and at least a portion of the remainder, tungsten. This alloy exhibits excellent biocompatibility and yet has sufficient strength and ductility to be wound into coils of primary and secondary shape and will retain those shapes upon placement of the vaso-occlusive device in the human body. The diameter of the wire typically making up the coils is often in a range of 0.005 and 0.050 inches, preferably between about 0.001 and about 0.003 inches in diameter.

Desirably, the mechanical occlusive devices are associated with some amount of a polymeric material, which may be comprised of a wide variety of materials. Synthetic and natural polymers, such as polyurethanes (including copolymers with soft segments containing esters, ethers and carbonates), ethers, acrylates (including cyanoacrylates), olefins (including polymers and copolymers of ethylene, propylene, butenes, butadiene, styrene, and thermoplastic olefin elastomers), polydimethyl siloxane-based polymers, polyethyleneterephthalate, cross-linked polymers, non-cross linked polymers, rayon, cellulose, cellulose derivatives such nitrocellulose, natural rubbers, polyesters such as lactides, glycolides, caprolactones and their copolymers and acid derivatives, hydroxybutyrate and polyhydroxyvalerate and their copolymers, polyether esters such as polydioxinone, anhydrides such as polymers and copolymers of sebacic acid, hexadecandioic acid and other diacids, orthoesters may be used. In a preferred embodiment, the polymeric filament comprises suture materials that have already been approved for use in wound healing in humans.

When a blood vessel is catheterized, blood often refluxes into the distal end of catheter. Since the polymer of our inventive composition precipitates as the solvent mixes with blood, a polymer solution injected through a catheter could precipitate in the catheter. In such an event, the inventive polymer solution likely would not reach the treatment site. Thus, it is highly desirable to separate the inventive polymer solution from the blood during the period of its delivery through the catheter. A plug of a “barrier solvent” is suitable for such separation. Ideally, the barrier solvent is miscible neither with blood nor with the polymer solution. However, many such immiscible solvents would be expected to be toxic to the body. Consequently, an alternative is to use a less effective but nonetheless suitable solvent system, e.g., a partially miscible solvent system, to separate the polymer solution from the blood. A 20-30% aqueous ethanol solution is effective as such a barrier.

When using the auxiliary mechanical occlusive devices, those devices should be first introduced to the chosen site using the procedure outlined below. This procedure may be used in treating a variety of maladies. For instance, in treatment of an aneurysm, the aneurysm itself may be filled with the mechanical devices prior to introducing the inventive composition. Shortly after the mechanical devices and the inventive composition are placed within the aneurysm, an emboli begins to form and, at some later time, is at least partially replaced by neovascularized collagenous material formed around the vaso-occlusive devices.

In using the mechanical occlusive devices, a selected site is reached through the vascular system using a collection of specifically chosen catheters and guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter as a unit. Once the tip of the guidewire reaches the end of the guiding catheter, it is then extended using fluoroscopy, by the physician to the site to be treated using the vaso-occlusive devices of this invention. During the trip between the treatment site and the guide catheter tip, the guidewire is advanced for a distance and the neurovascular catheter follows. Once both the distal tip of the neurovascular catheter and the guidewire have reached the treatment site, and the distal tip of that catheter is appropriately situated, e.g., within the mouth of an aneurysm to be treated, the guidewire is then withdrawn. The neurovascular catheter then has an open lumen to the outside of the body. The devices of this invention are then pushed through the lumen to the treatment site. They are held in place variously because of their shape, size, or volume. These concepts are described in the Ritchart et al patent as well as others. Once the vaso-occlusive devices are situated in the vascular site, the embolism forms.

The mechanical or solid vaso-occlusion device may be used as a kit with the inventive polymeric precursor composition.

Modifications of the procedure and device described above, and the methods of using them in keeping with this invention will be apparent to those having skill in this mechanical and surgical art. These variations are intended to be within the scope of the claims that follow.

We claim as our invention:

1. A precursor composition for forming a biologically active anatomical occlusion in an anatomical cavity, comprising:
a) a biodegradable, polymeric occlusion-forming component; and
b) a biologically active component,
wherein said precursor composition forms a biologically active occlusion mass when introduced into the anatomical cavity.

2. The precursor composition of claim 1 wherein the polymeric occlusion-forming component comprises a biodegradable polymer dissolved in a biologically tolerated solvent, said polymer precipitating from said precursor composition when introduced into the anatomical cavity.

3. The precursor composition of claim 1 wherein the polymeric occlusion-forming component comprises a biodegradable component reactively forming a polymer mass when introduced into the anatomical cavity.

4. The precursor composition of claim 1 wherein the polymeric occlusion-forming component comprises a biodegradable polymer selected from biodegradable polyesters.

5. The precursor composition of claim 4 wherein the biodegradable polyester are selected from polyglycolic acids, polylactic acids, polycaprolactone, and their copolymers and copolymers with trimethylene carbonate.

6. The precursor composition of claim 4 wherein the biodegradable polymer is selected from polyhydroxybutyrate and polyhydroxyvalerate and their copolymers.

7. The precursor composition of claim 4 wherein the biodegradable polymer is a polyanhydride.

8. The precursor composition of claim 1 further comprising a biologically tolerated solvent.

9. The precursor composition of claim 1 wherein said biologically active component is selected from the group consisting of collagen, fibrinogen, vitronectin, plasma proteins, growth factors, synthetic peptides of these and other proteins having attached RGD (arginine-glycine-aspartic acid) residues at one or both termini, cell adhesion peptides, oligonucleotides, full or partial DNA constructs, natural or synthetic phospholipids, polymers with phosphorylcholine functionality, and polynucleotide sequences encoding peptides (e.g., genes) involved in wound healing or promoting cellular attachment.

10. The precursor composition of claim 1 wherein said biologically active component is selected from the group consisting of genes, growth factors, biomolecules, peptides, oligonucleotides, members of the integrin family, RGD-containing sequences, and oligopeptides.

11. The precursor composition of claim 10 wherein said oligopeptides are selected from the group consisting of fibronectin, laminin, bitronectin, hyaluronic acid, silk-elastin, elastin, fibrinogen, and other basement membrane proteins.

12. A solid, bioactive, biodegradable polymeric occlusive mass.

13. The occlusive mass of claim 12 wherein the mass contains a biodegradable polymer selected from biodegradable polyesters.

14. The occlusive mass of claim 12 wherein the biodegradable polyesters are selected from polyglycolic acids, polylactic acids, polycaprolactone, and their copolymers and copolymers with trimethylene carbonate.

15. The occlusive mass of claim 14 wherein the mass contains a biodegradable polymer selected from polyhydroxybutyrate and polyhydroxyvalerate and their copolymers.

16. The occlusive mass of claim 14 wherein the mass contains a polyanhydlide.

17. The occlusive mass of claim 12 wherein the mass contains a biologically active component selected from the group consisting of collagen, fibrinogen, vitronectin, plasma proteins, growth factors, synthetic peptides of these and other proteins having attached RGD (arginine-glycine-aspartic acid) residues at one or both termini, cell adhesion peptides, oligonucleotides, full or partial DNA constructs, natural or synthetic phospholipids, polymers with phosphorylcholine functionality, and polynucleotide sequences encoding peptides (e.g., genes) involved in wound healing or promoting cellular attachment.

18. The occlusive mass of claim 12 wherein the mass contains a biologically active component selected from the group consisting of genes, growth factors, biomolecules, peptides, oligonucleotides, members of the integrin family, RGD-containing sequences, and oligopeptides.

19. The occlusive mass of claim 18 wherein said oligopeptides are selected from the group consisting of fibronectin, laminin, bitronectin, hyaluronic acid, silk-elastin, elastin, fibrinogen, and other basement membrane proteins.

20. A kit for forming a composite biologically active anatomical occlusion in an anatomical cavity, comprising:
a) at least one solid vaso-occlusive device, and
b.) a liquid precursor composition comprising:
i.) a biodegradable, polymeric occlusion-forming component; and
ii.) a biologically active component,
wherein said liquid precursor composition forms a biologically active occlusion mass when introduced into the anatomical cavity.

21. The kit of claim 20 wherein said at least one solid vaso-occlusive device comprises a coil.

22. The kit of claim 21 wherein said biodegradable, polymeric occlusion-forming component comprises a biodegradable polymer selected from biodegradable polyesters.

23. The kit of claim 22 wherein said biodegradable polyesters are selected from polyglycolic acids, polylactic acids, polycaprolactone, and their copolymers and their copolymers with trimethylene carbonate.

24. The kit of claim 22 wherein the biodegradable polymer is selected from polyhydroxybutyrate and polyhydroxyvalerate and their copolymers.

25. The kit of claim 22 wherein the biodegradable polymer is a polyanhydride.

26. The kit of claim 20 wherein the liquid precursor composition further comprises a biologically tolerated solvent.

27. The kit of claim 20 wherein said biologically active component is selected from the group consisting of collagen, fibrinogen, vitronectin, plasma proteins, growth factors, synthetic peptides of these and other proteins having attached RGD (arginine-glycine-aspartic acid) residues at one or both termini, cell adhesion peptides, oligonucleotides, full or partial DNA constructs, natural or synthetic phospholipids, polymers with phosphorylcholine functionality, and polynucleotide sequences encoding peptides (e.g., genes) involved in wound healing or promoting cellular attachment.

28. The kit of claim 20 wherein said biologically active component is selected from the group consisting of genes, growth factors, biomolecules, peptides, oligonucleotides, members of the integrin family, RGD-containing sequences, and oligopeptides.

29. The kit of claim 28 wherein said oligopeptides are selected from the group consisting of fibronectin, laminin, bitronectin, hyaluronic acid, silk-elastin, elastin, fibrinogen, and other basement membrane proteins

30. A procedure for at least partially filling an anatomical cavity comprising the steps of:
a.) introducing the precursor composition of claim 1 into said anatomical vessel,
b.) precipitating said biodegradable, polymeric occlusion-forming component and said biologically active component into said biologically active occlusion mass in said anatomical cavity.

31. The procedure of claim 30 further comprising the prior step of introducing a mechanical vaso-occlusive device into said anatomical cavity.
